(19) United States
(12) Patent Application Publication
(10) Pub. No.: US 2023/0416724 A1
(43) Pub. Date: Dec. 28, 2023

(54) Lysis Buffers Comprising Cyanate or Thiocyanate and a Detergent

(71) Applicant: Purigen Biosystems, Inc., Pleasanton, CA (US)

(72) Inventors: Nathan P. Hoverter, San Jose, CA (US); Lewis A. Marshall, Oakland, CA (US); Klint A. Rose, Oakland, CA (US); Debannita Datta, San Ramon, CA (US); Douglas Fadrosh, Danville, CA (US)

(21) Appl. No.: 18/212,882

(22) Filed: Jun. 22, 2023

Related U.S. Application Data
(60) Provisional application No. 63/355,369, filed on Jun. 24, 2022.

(51) Int. Cl. C12N 15/10; G01N 27/447

(52) U.S. Cl. CPC C12N 15/101; G01N 27/44721

(57) ABSTRACT

The present disclosure provides cell lysis buffers and method of use. Cell lysis buffers may comprise an aqueous solution comprising a buffering agent, a detergent, and cyanate or thiocyanate. The cell lysis buffers may be free or substantially free of urea and/or carbonic acid. Such lysis buffers may be used to lyse cells, e.g., in tissues. The cell lysis buffers may be suitable for use with isotachophoresis.

### CROSS-REFERENCE TO RELATED APPLICATIONS

Pursuant to 35 U.S.C. § 119 (e), this application claims priority to the filing date of U.S. Provisional Patent Application Ser. No. 63/355,369 filed Jun. 24, 2022; the disclosure of which application is incorporated herein by reference in their entirety.

### BACKGROUND

Lysis buffers generally refer to buffer solutions used to lyse cells and release their contents for analysis. Lysis buffers may contain a buffering agent, such as Tris, and an ionic salt, such as NaCl, to regulate the pH and/or osmolarity of a lysate. Detergents, such as Triton X-100 or SDS, may be added to dissolve membrane structures.

### SUMMARY

In an aspect, the present disclosure provides a composition, comprising a) a buffer in aqueous solution; b) a detergent; and c) about 5 mM to about 100 mM cyanate or thiocyanate; wherein a relative concentration of the cyanate or the thiocyanate to urea in the composition is at least about 1:800, at least about 1:600, at least about 1:400, at least about 1:200, at least about 1:10, at least about 1:1, at least about 100:1, at least about 10,000:1, or at least about 1,000,000:1.

In some cases, the detergent is an ionic detergent. In some examples, the ionic detergent is a cationic, anionic, zwitterionic or amphoteric detergent. In some cases, the detergent is a surfactant. In some cases, the detergent is a non-ionic detergent. In some cases, the relative concentration is a molar concentration (e.g., moles/L, moles/ml). In some cases, the relative concentration is a mass concentration (e.g., mg/L, mg/ml, g/L, etc.).

In some embodiments, the composition comprises no more than about 500 mM urea, no more than about 10 mM urea, no more than about 2 mm urea, no more than about 100 nM urea, or no more than about 1 nanomolar urea. In some embodiments, the composition is free or essentially free of urea. In some embodiments, the composition is free or essentially free of guanidinium. In some embodiments, the composition further comprises about 0.0001 mM to 10 mM, about 0.001 mM to 10 mM, about 0.01 mM to 10 mM, about 0.1 mM to 10 mM, about 1 mM to 10 mM, about 10 mM to 25 mM cyanate or thiocyanate, about 25 mM to 50 mM cyanate or thiocyanate, or about 50 mM to 100 mM cyanate or thiocyanate. In some embodiments, the cyanate or the thiocyanate comprises sodium cyanate, sodium thiocyanate, potassium cyanate, or potassium thiocyanate. In some embodiments, the composition further comprises an ionic salt. In some embodiments, the ionic salt comprises a chloride salt. In some embodiments, the chloride salt comprises sodium chloride (NaCl) or potassium chloride (KCl). In some embodiments, the ionic salt is present at about 10 mM to 40 mM. In some embodiments, the buffer comprises phosphate, Tris, Bis-Tris, e-aminocaproic acid, or HEPES. In some embodiments, the composition further comprises about 10 mM to 200 mM Tris-chloride. In some embodiments, the composition further comprises about 10 mM to 200 mM Bis-Tris-chloride. In some embodiments, the composition further comprises about 10 mM to 200 mM e-aminocaproic acid. In some embodiments, the aqueous solution has a pH between about 3.5 and about 9.0. In some embodiments, the aqueous solution has a pH between about 6.5 and about 8.5. In some embodiments, the buffer has a concentration between about 10 mM and about 200 mM. In some embodiments, the buffer has a concentration between about 10 mM and about 40 mM. In some embodiments, the ionic detergent comprises a dodecyl sulfate or ethyl trimethylammonium bromide (“ETMAB”). In some embodiments, the dodecyl sulfate comprises sodium dodecyl sulfate. In some embodiments, the composition further comprises a reducing agent. In some embodiments, the reducing agent comprises DTT (dithiothreitol), DTE (dithioerythritol), L-glutathione (GSH), TCEP (Tris (2-Carboxyethyl) phosphine hydrochloride), or beta-mercaptoethanol. In some embodiments, the composition further comprises a protease. In some embodiments, the protease comprises proteinase K. In some embodiments, for each 1 ul to 20 ul of the composition, the protease has 0.8 activity units to 16 activity units or is present in an amount of 20 ug-400 ug. In some embodiments, the composition further comprises less than 100 mM or less than 2 mM guanidinium. In some embodiments, the composition further comprises a nucleic acid. In some embodiments, the composition further comprises a cell lysate. In some embodiments, the composition further comprises formalin fixed paraffin embedded tissue.

In another aspect, the present disclosure provides a method comprising: a) providing a mixture comprising an amount of cells and a composition according to any one of claims 1-29, and b) incubating the mixture for a time sufficient to lyse the cells and digest endogenous proteins, to produce a cell lysate mixture.

In some embodiments, the cells are comprised in FFPE or fresh/frozen tissue. In some embodiments, the tissue comprises a fine needle aspirate or a core needle biopsy. In some embodiments, the tissue is derived from lung, heart kidney, liver, colon breast, lung, brain, or skin. In some embodiments, the tissue is Formalin Fixed Paraffin Embedded (“FFPE”) tissue. In some embodiments, the amount of cells is about 0.1 mg to about 25 mg, and wherein the composition is about 50 ul to about 2000 ul. In some embodiments, the cells are incubated between 37° C. and 56° C. In some embodiments, the time is between 10 minutes and 24 hours, between about 5 minutes and about 30 minutes, or about 15 minutes) to isolate RNA, or about 1 hour to about 5 hours, or about 3 hours to isolate DNA. In some embodiments, the method further comprises centrifuging the mixture after the incubating, and collecting supernatant from the centrifuged mixture. In some embodiments, the method further comprises contacting the mixture with a nuclease. In some embodiments, the nuclease is a DNase or an RNase. In some embodiments, the method further comprises: c) performing isotachophoresis (“ITP”) on the cell lysate mixture after the incubating. In some embodiments, performing the ITP comprises: (a) loading into a fluidic device: (i) the cell lysate mixture; (ii) a trailing electrolyte buffer comprising trailing electrolyte ions with an effective mobility having a magnitude lower than a magnitude of an effective mobility of nucleic acids in the cell lysate mixture; and (iii) a leading electrolyte buffer comprising first leading electrolyte ions, with a second effective mobility, wherein the second effective mobility has a magnitude greater than the magnitude of said effective mobility of said nucleic acids in the cell lysate mixture; and (b) applying an electric field across the fluidic device to conduct isotachophoresis with the trailing electrolyte ions, said nucleic acids, and the first leading electrolyte. In some embodiments, the method further comprises, before performing ITP, contacting the composition with a quenching solution comprising a non-ionic detergent. In some embodiments, the non-ionic detergent comprises a hydrophilic polyethylene oxide chain (e.g., octylphenoxypolyethoxyethanol (“IGEPAL CA-630” or “Nonidet P-40”), a polysorbate (e.g., Tween-20 or Tween 80), a Triton X (e.g., Trigon X 100 or Triton X 114), octyl glucoside, octyl thioglucoside, Brij, SPAN, or MEGA. In some embodiments, the method further comprises, while performing the ITP, measuring conductivity of the trailing electrolyte with an infrared sensor. In some embodiments, the cell lysate mixture used in the ITP is essentially free of carbonic acid. In some embodiments, the nucleic acid is RNA.

In another aspect, the present disclosure provides a kit comprising: (a) a container containing a composition disclosed herein; (b) trailing electrolyte buffer comprising trailing electrolyte ions with an effective mobility having a magnitude lower than a magnitude of an effective mobility of nucleic acids; and (c) a leading electrolyte buffer comprising first leading electrolyte ions, with a second effective mobility, wherein the second effective mobility has a magnitude greater than the magnitude of the effective mobility of nucleic acids.

In some embodiments, the kit further comprises a container containing a quenching solution comprising a non-ionic detergent. In some embodiments, the leading electrolyte ions comprise chloride, and wherein the trailing electrolyte ions comprise caproic acid, Hepes, 3-(N-morpholino)propanesulfonic acid (“MOPS”), 2-(N-morpholino)ethanesulfonic acid (“MES”), ascorbic acid, or vanilic acid.

In another aspect, the present disclosure provides an article of manufacture comprising a fluidic device comprising a fluidic channel comprising: (A) a test sample, wherein the test sample comprises a composition disclosed herein and nucleic acids, (B) a trailing electrolyte (“TE”) buffer comprising trailing electrolyte ions with an effective mobility having a magnitude lower than a magnitude of an effective mobility of the nucleic acids, and (C) a leading electrolyte (“LE”) buffer comprising leading electrolyte ions, with a second effective mobility, wherein the second effective mobility has a magnitude greater than the magnitude of the effective mobility of the nucleic acids.

In some embodiments, the test sample further comprises a non-ionic detergent.

In another aspect, the present disclosure provides a system comprising: a) a fluidic device comprising a fluidic channel comprising: (A) a test sample, wherein the test sample comprises a composition disclosed herein and nucleic acids, (B) a trailing electrolyte (“TE”) buffer comprising trailing electrolyte ions with an effective mobility having a magnitude lower than a magnitude of an effective mobility of the nucleic acids, and (C) a leading electrolyte (“LE”) buffer comprising leading electrolyte ions, with a second effective mobility, wherein the second effective mobility has a magnitude greater than the magnitude of the effective mobility of the nucleic acids; b) an instrument comprising a power source, and an anode and a cathode attached to power source and positioned to provide a voltage difference between the anode and the cathode, wherein the anode is in electrical communication with the TE buffer, and wherein the cathode is in electrical communication with the LE buffer.

In some embodiments, the test sample further comprises a non-ionic detergent.

In another aspect, the present disclosure provides a method of preparing a composition, comprising: a) providing a buffer and a detergent, wherein the buffer and the ionic detergent are mixed or unmixed; b) providing an amount of cyanate or thiocyanate that is substantially free of urea; and c) mixing together in an aqueous solution, the buffer, the detergent, and the amount of the cyanate or the thiocyanate that is substantially free of urea, wherein the amount is sufficient to produce a concentration of about 5 mM to about 100 mM of cyanate or thiocyanate. In some cases, the detergent is an ionic detergent. In some examples, the ionic detergent is a cationic, anionic, zwitterionic or amphoteric detergent. In some cases, the detergent is a surfactant. In some cases, the detergent is a non-ionic detergent. In some cases, the relative concentration is a molar concentration.

In some embodiments, the amount of the cyanate or the thiocyanate that is substantially free of urea is sufficient to produce a relative concentration of cyanate or thiocyanate to urea in the composition of at least about 1:10000, at least about 1:1000; 1:800, at least about 1:600, at least about 1:400, at least about 1:200, at least about 1:10, at least about 1:1, at least about 100:1, at least about 10,000:1, or at least about 1,000,000:1. In some embodiments, the amount of the cyanate or the thiocyanate that is substantially free of urea is sufficient to produce a relative concentration of cyanate or thiocyanate to urea in the composition of at least about 1:800, at least about 1:600, or at least about 1:400, In some embodiments, the composition comprises no more than about 500 mM urea, no more than about 10 mM urea, no more than about 2 mm urea, no more than about 100 nM urea, no more than about 80 nM urea, no more than about 70 nM urea, no more than about 50 nM urea, no more than about 20 nM urea, no more than about 10 nM urea, or no more than about 1 nanomolar urea. In some embodiments, the composition is free or essentially free of urea. In some embodiments, the composition is free or essentially free of guanidinium. In some embodiments, the composition comprises about 10 mM to 25 mM cyanate or thiocyanate, about mM to 50 mM cyanate or thiocyanate, or about 50 mM to 100 mM cyanate or thiocyanate. In some embodiments, the cyanate or the thiocyanate comprises sodium cyanate, sodium thiocyanate, potassium cyanate, or potassium thiocyanate. In some embodiments, the method further comprises adding an ionic salt to the aqueous solution. In some embodiments, the ionic salt comprises a chloride salt. In some embodiments, the chloride salt comprises sodium chloride (NaCl) or potassium chloride (KCl). In some embodiments, the ionic salt is present at about 10 mM to 40 mM. In some embodiments, the buffer comprises phosphate, Tris, Bis-Tris, e-aminocaproic acid, or HEPES. In some embodiments, the buffer comprises about 10 mM to 200 mM Tris-chloride. In some embodiments, the buffer comprises about 10 mM to 200 mM Bis-Tris-chloride. In some embodiments, the buffer comprises about 10 mM to 200 mM e-aminocaproic acid. In some embodiments, the aqueous solution has a pH between about 3.5 and about 9.0. In some embodiments, the aqueous solution has a pH between about 6.5 and about 8.5. In some embodiments, the buffer has a concentration between about 10 mM and about 200 mM. In some embodiments, the buffer has a concentration between about 10 mM and about 40 mM. In some embodiments, the ionic detergent comprises a dodecyl sulfate or ethyl trimethylammonium bromide (“ETMAB”). In some embodiments, the dodecyl sulfate comprises sodium dodecyl sulfate. In some embodiments, the method further comprises adding a reducing agent to the aqueous solution. In some embodiments, the reducing agent comprises DTT (dithiothreitol), DTE (dithioerythritol), L-glutathione (GSH), TCEP (Tris (2-Carboxyethyl) phosphine hydrochloride), or beta-mercaptoethanol. In some embodiments, the method further comprises adding a protease to the aqueous solution. In some embodiments, the protease comprises proteinase K. In some embodiments, for each 1 ul to 20 ul of the composition, the protease has 0.8 activity units to 16 activity units or is present in an amount of 20 ug to 400 ug. In some embodiments, the composition comprises less than 100 mM or less than 2 mM guanidinium. In some embodiments, the method further comprises contacting the composition with a nucleic acid. In some embodiments, the method further comprises contacting the composition with a cell lysate. In some embodiments, the method further comprises contacting the composition with formalin fixed paraffin embedded tissue. In some embodiments, the method further comprises contacting the composition with an amount of cells to form a mixture, and incubating the mixture for a time sufficient to lyse the cells and digest endogenous proteins, to produce a cell lysate mixture. In some embodiments, the method further comprises contacting the composition with a nuclease. In some embodiments, the nuclease is a DNase or an RNase. In some embodiments, the buffer and the ionic detergent are mixed in a). In some embodiments, the buffer and the ionic detergent are unmixed in a).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an isotachophoresis (ITP) process.

FIG. 2 shows an example of a cartridge for ITP comprising fluidic channels.

FIG. 3 shows fluidic architecture of an example of a fluidic circuit for ITP.

FIG. 4 shows an example of an instrument for ITP loaded with a cartridge.

FIG. 5A-5B show an example of impact of Cyanate on DNA degradation where Cyanate prevents DNA degradation.

FIG. 6 shows an example of Post-Development HI Cyanate Benchmark comparison between different conditions. Described conditions had 6-7× yield than Qiagen Mini (eluted with ul) (Data not shown).

FIG. 7 shows an example of Post-Development HI Cyanate Benchmark—qPCR where cyanate had ˜3× amplifiable yield than Qiagen Mini.

FIG. 8 shows an example where there are no obvious signs of inhibition in qBiomarker assay.

FIG. 9 shows an example of effects of cyanate on qPCR inhibition.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### I. Introduction

Lysis buffers generally refer to buffer solutions used to lyse cells and release their contents for analysis. Lysis buffers may contain a buffering agent, such as Tris, and an ionic salt, such as NaCl, to regulate the pH and/or osmolarity of a lysate. Detergents, such as Triton X-100 or SDS, may be added to dissolve membrane structures.

Enzyme inhibitors may be added to the buffer to inhibit the activity of enzymes that modify or destroy target macromolecules. For example, DNase inhibitors may be added to inhibit digestion of DNA. RNase inhibitors may be added to inhibit the digestion of RNA. Protease inhibitors may be added to inhibit digestion of proteins. Phosphatase inhibitors may be added to inhibit phosphorylation of proteins.

Guanidinium thiocyanate may be used for cell lysis. For example, guanadinium may be used at extremely high concentration (e.g., greater than about 1 molar (M)). As another example, urea may be used at high concentration. However, these agents may interfere with certain downstream modes of preparation and analysis, such as isotachophoresis (“ITP”).

The present disclosure provides methods, systems, compositions, and kits comprising lysis buffers comprising a detergent and cyanate or thiocyanate. Such lysis buffers may exhibit improved performance as nuclease inhibitors, thereby preserving nucleic acids in a cell lysate. Lysis buffers of the present disclosure may be prepared at least in part by reducing, eliminating, or essentially eliminating urea from lysis buffers, while maintaining a desired concentration of cyanate or thiocyanate, which may improve performance of a cell lysate, e.g., for applications such as isotachophoresis (“ITP”). Urea may uncontrollably degrade into cyanate and carbonic acid. Ionic species, such as carbonic acid, may interfere with isotachophoresis. Therefore, reducing such ionic species in lysis buffers improves the performance of applications using cell lysates, such as ITP. Accordingly, the present disclosure provides methods of performing ITP comprising providing cell lysate samples for ITP in which the concentration of interfering substances such as carbonic acid is reduced, for example, by reducing, eliminating, or essentially eliminating interfering agents, such as urea and/or guanidinium, from cell lysis buffers.

### II. Lysis Buffers Containing Cyanate or Thiocyanate

In some embodiments, the present disclosure provides lysis solutions comprising an aqueous buffer, a detergent, and cyanate or thiocyanate (e.g., at a concentration of about 5 millimolar (mM) to about 100 mM). In some embodiments, the lysis solutions are free of urea, essentially free of urea, or, to the extent they comprise urea, have a relative concentration of cyanate or thiocyanate to urea of at least about 1:200 (e.g., at least about 20 mM cyanate or thiocyanate to about 4 molar (M) urea). In some embodiments, lysis solutions comprise an ionic salt, a reducing reagent, and an enzyme inhibitor. In some embodiments, the lysis solutions may be essentially free of carbonic acid or guanidinium. In some embodiments, the present disclosure provides lysis solutions that inhibit degradation of nucleic acids and produce mixtures that are more compatible with isotachophoresis.

A. Aqueous Buffers

In some embodiments, the present disclosure provides lysis buffers comprising an aqueous solution comprising a buffering agent. The buffer may be any buffer suitable for use in lysis solutions. These include, for example, without limitation, phosphate, Tris, Bis-Tris, e-aminocaproic acid, or HEPES. The buffer may have a concentration of between about 10 millimolar (mM) and about 200 mM. The pH of the solution may depend on the particular application. However, pH may be between about pH 3.5 and about pH 9.0. For example, the pH may be between about 3.5 and about 4.0, between about 4.0 and about 4.5, between about 4.5 and about 5.0, between about 5.0 and about 5.5, between about 5.5 and about 6.0, between about 6.0 and about 6.5, between about 6.5 and about 7.0, between about 7.0 and about 7.5, between about 7.5 and about 8.0, between about 8.0 and about 8.5, between about 8.5 and about 9.0. For example, the pH may be about 3.5, about 4.0, about 4.5, about 5.0, about 5.5, about 6.0, about 6.5, about 7.0, about 7.5, about 8.0, about 8.5, or about 9.0.

B. Detergents

In some embodiments, the present disclosure provides lysis solutions comprising detergents. In some cases, the detergent is an ionic detergent. In some cases, the detergent is a non-ionic detergent. In some examples, the ionic detergent is a cationic, anionic, zwitterionic or amphoteric detergent. In some cases, the detergent is a surfactant. The ionic detergent may aid in disruption of cell membranes. Examples of ionic detergents include dodecyl sulfates, (e.g., sodium dodecyl sulfate) or ethyl trimethylammonium bromide (“ETMAB”).

Ionic detergents may be present at concentrations of about 0.1% w/v to about 10% w/v, such as about 0.1% w/v, about 0.3% w/v, about 0.5% w/v, about 1% w/v, about 2% w/v, about 3% w/v, about 4% w/v, about 5% w/v, about 6% w/v, about 7% w/v, about 8% w/v, about 9% w/v, or about 10% w/v.

C. Cyanate, Urea, and Carbonic Acid

Cyanate, as used herein, generally refers to any salt or ester of cyanic acid. Cyanate may refer to isocyanate. Any suitable form of cyanate may be used. This includes, for example, sodium cyanate, potassium cyanate, and silver cyanate. Thiocyanate, as used herein, generally refers to any salt or ester of thiocyanic acid. In some embodiments, thiocyanate be used instead of cyanate in lysis buffers. Cyanide, as used herein, generally refers to any compound containing the C≡N radical or the C≡N⁻¹ anion.

In some embodiments, the present disclosure provides lysis buffers comprising between about 5 mM and about 100 mM cyanate or thiocyanate (e.g., about 5 mM, about 10 mM, about 20 mM, about 30 mM, about 40 mM, about 50 mM, about 60 mM, about 70 mM, about 80 mM, about 90 mM, or about 100 mM). Cyanate or thiocyanate may be used for inhibition of ribonuclease and/or deoxyribonuclease activity. In some embodiments, the present disclosure provides lysis solutions comprising controlled amounts of urea and/or carbonic acids, if present.

Urea may be a component of lysis buffers. However, urea may degrade into other components including cyanate and carbonic acid. Cyanate may be used as a nuclease inhibitor. However, carbonic acid may interfere with ITP. Furthermore, the levels of these degradation components in a urea sample may depend on factors such as the age of the urea solution. Therefore, in any urea sample, these species may represent uncontrolled and/or unpredictable variables. Accordingly, the present disclosure provides compositions in which variables, such as the levels of degradation components, are controlled.

In some embodiments, the concentration of cyanate in a lysis buffer is controlled by adding or introducing known quantities of cyanate and/or reducing the amount or concentration of urea. Reducing the amount or concentration of urea may also reduce the amount of its degradation products, such as carbonic acid.

Accordingly, the present disclosure provides lysis buffers comprising between about mM and 100 mM cyanate or thiocyanate. For example, the lysis buffer may comprise between about 10 mM and about 25 mM cyanate or thiocyanate, between about 25 mM and about 50 mM cyanate or thiocyanate, or between about 50 mM and about 100 mM cyanate or thiocyanate. As another example, the lysis buffer may comprise about 5 mM, about 10 mM, about 20 mM, about 30 mM, about 40 mM, about 50 mM, about 60 mM, about 70 mM, about 80 mM, about 90 mM, or about 100 mM cyanate or thiocyanate.

In some embodiments, if urea is present in the lysis buffer, it may present in an amount or concentration such that the ratio of cyanate or thiocyanate to urea is at least about 1:200. For example, the relative concentrations may be about 20 mM cyanate or thiocyanate to about 4 molar urea. In some embodiments, the lysis buffer is free or essentially free of urea. As used herein, a lysis buffer is “essentially free of urea” if the concentration of urea in the solution is no more than about 2 mM. For example, a lysis buffer essentially free of urea may have a ratio of about 20 mM cyanate or thiocyanate to about 2 mM urea, for a relative concentration of about 10:1 cyanate:urea.

The ratio of the concentrations of cyanate:urea may be at least about 1:800, at least about 1:600, at least about 1:400, at least about 1:200, at least about 1:10, at least about 1:1, at least about 100:1, at least about 10,000:1, or at least about 1,000,000:1. Alternatively, given a cyanate concentration of between about 5 mM and 100 mM, the concentration of urea in the lysis buffer may be no more than about 500 mM, no more than about 10 mM, no more than about 2 mm, no more than about 100 nM, or no more than about 1 nanomolar.

In some embodiments, lysis buffers comprises limited amounts of carbonic acid, are free or carbonic acid, or are essentially free of carbonic acid. For example, the amount of carbonic acid in the lysis buffer may be no more than about 20 mM, no more than about 15 mM, no more than about 10 mM, no more than about 5 mM, no more than about 4 mM, more than about 3 mM, more than about 2 mM, or substantially free of carbonic acid.

In some embodiments, lysis buffers are free or essentially free (e.g., no more than about 2 mM) of guanidinium ions. For example, the amount of guanidinium in the lysis buffer may be no more than about 20 mM, no more than about 15 mM, no more than about 10 mM, no more than about 5 mM, no more than about 4 mM, more than about 3 mM, more than about 2 mM, or substantially free of guanidinium.

D. Ionic Salts

In some embodiments, the present disclosure provides lysis buffers comprising ionic salts. Examples of salts include NaCl, KCl, and (NH₄)₂SO₄. Salts may be present at a concentration between about 10 mM and about 150 mM. For example, the lysis buffer may comprise about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, or about 150 mM salt. In some embodiments, lysis buffers are free or essentially free of salt.

E. Reducing Agents

In some embodiments, the present disclosure provides lysis buffers comprising reducing agents. The reducing agent may be, for example, dithiothreitol (“DTT”), Dithioerythritol (“DTE”), and 2-Mercaptoethanol (“2-Me”). DTE and/or DTT may be present in concentrations of about 1 mM to 50 mM, (e.g., between about 5 mM and 15 mM, e.g., about 10 mM). For example, the lysis buffer may comprise about 1 mM, about 2 mM, about 3 mM, about 4 mM, about 5 mM, about 6 mM, about 7 mM, about 8 mM, about 9 mM, about 10 mM, about 20 mM, about 30 mM, about 40 mM, or about 50 mM DTE and/or DTT. 2-Me may be present in concentrations of about 0.05% w/v.

F. Chelating Agents

In some embodiments, the present disclosure provides lysis buffers comprising chelating agents. Examples of chelating agents include EDTA (Ethylenediaminetetraacetic acid), EGTA (ethylene glycol-bis((3-aminoethyl ether)-N,N,N′,N′-tetraacetic acid), and ethylenediamine. Chelating agents may be present in concentrations of about 1 mM to 10 mM. For example, the lysis buffer may comprise about 1 mM, about 2 mM, about 3 mM, about 4 mM, about 5 mM, about 6 mM, about 7 mM, about 8 mM, about 9 mM, or about 10 mM chelating agents.

G. Enzymes

In some embodiments, the present disclosure provides lysis buffers comprising enzymes that degrade non-target classes of molecules. For example, in methods of isolating nucleic acids, proteases may be included to degrade proteins. Examples of proteases include proteinase K, protease type XIV, protease from Streptomyces griseus, papain and pepsin. In methods of isolation DNA, agents that degrade RNA may be included in the buffer. In methods of isolating RNA, agents that degrade DNA may be included in the buffer. This may include, for example, a DNase. Commercially available DNase preparations may include, for example, DNase I (Sigma-Aldrich), Turbo DNA-free (ThermoFisher) or RNase-Free DNase (Qiagen). This may include, for example, an RNase. RNAses include RNAse (Qiagen), and RNAse I (ThermoFisher).

### III. Methods of Lysis

In some embodiments, the present disclosure provides lysis methods comprising contacting a sample comprising cells with a lysis buffer as disclosed herein, and incubating the mixture for a time sufficient to lyse the cells.

Samples comprising cells may include, for example, without limitation, cultured cells, tissue samples, FFPE (formalin fixed paraffin embedded) tissue, fresh/frozen tissue, tissue biopsy (e.g., fine needle aspirates and core needle biopsies). Examples of tissues may include, for example, without limitation, those from lung, heart kidney, liver, colon breast, lung, brain, or skin.

In some embodiments, the amount of cells in the sample is about 0.1 mg to about 25 mg, and the composition is about 50 ul to about 2000 ul. The mixture may be incubated at a temperature between about 37° C. and about 56° C. The incubation time may be between about 10 minutes and about 24 hours.

### IV. Methods of Purification

Nucleic acids liberated by cell lysis may be purified from the cell lysate by various suitable approaches.

A. Isotachophoresis

Nucleic acids liberated by cell lysis also may be further purified by isotachophoresis.

Isotachophoresis (ITP) may refer to an analytical method in which charged analytes are separated based on ionic mobility. It may be a form of electrophoresis. A sample comprising an analyte and contaminants may be placed in a fluidic channel containing a fast leading electrolyte (LE) and a slow trailing electrolyte (TE). The electrolytes may be chosen such that the analyte molecules have an ionic mobility between the leading electrolyte and the trailing electrolyte. In the case of nucleic acids, the leading electrolyte ions may comprise chloride and the trailing electrolyte ions may comprise, for example, caproic acid, Hepes, 3-(N-morpholino)propanesulfonic acid (“MOPS”), 2-(N-morpholino)ethanesulfonic acid (“MES”), ascorbic acid, glycine, tricine, TAPS, capric acid, propionic acid, cinnamic acid, and vanillic acid. After establishing a voltage gradient across the fluidic channel, the ions may separate, focusing the analyte molecules between the leading and trailing electrolytes. Contaminants may either migrate faster or slower than the leading and trailing electrolytes and are thereby separated from the analyte. Analyte molecules may be removed from the fluidic channel and subject to further analysis or use. For example, purified nucleic acid molecules may be sequenced or quantified or cloned into a desired cloning vehicle.

In some ITP methods, infrared sensing is used to determine the location of the sample in an ITP channel. Carbonic acid may interfere with such measurements. Therefore, lysis buffers low in carbonic acid may exhibit improved performance in this regard.

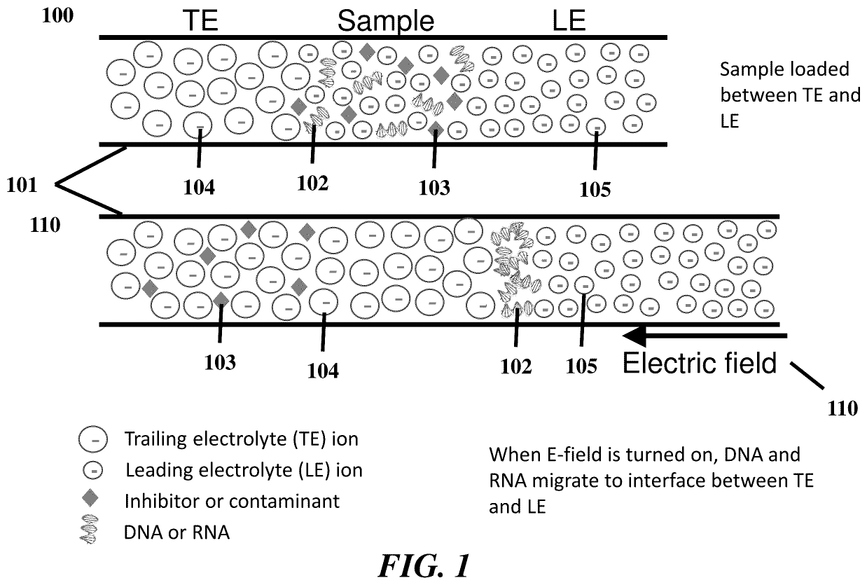

Referring to FIG. 1, in some embodiments of ITP, in the starting position 100, ITP fluidic channel 101, is loaded with trailing ions 104, leading ions, 105 and, between them a sample comprising nucleic acid molecules 102 and contaminants 103. After application of an electric field 110, leading ions 105 move more rapidly toward the electrode and trailing ions 104. Contaminant 103 migrates with the trailing ions in this example, while nucleic acids 102 and focused and purified between the leading trailing ions.

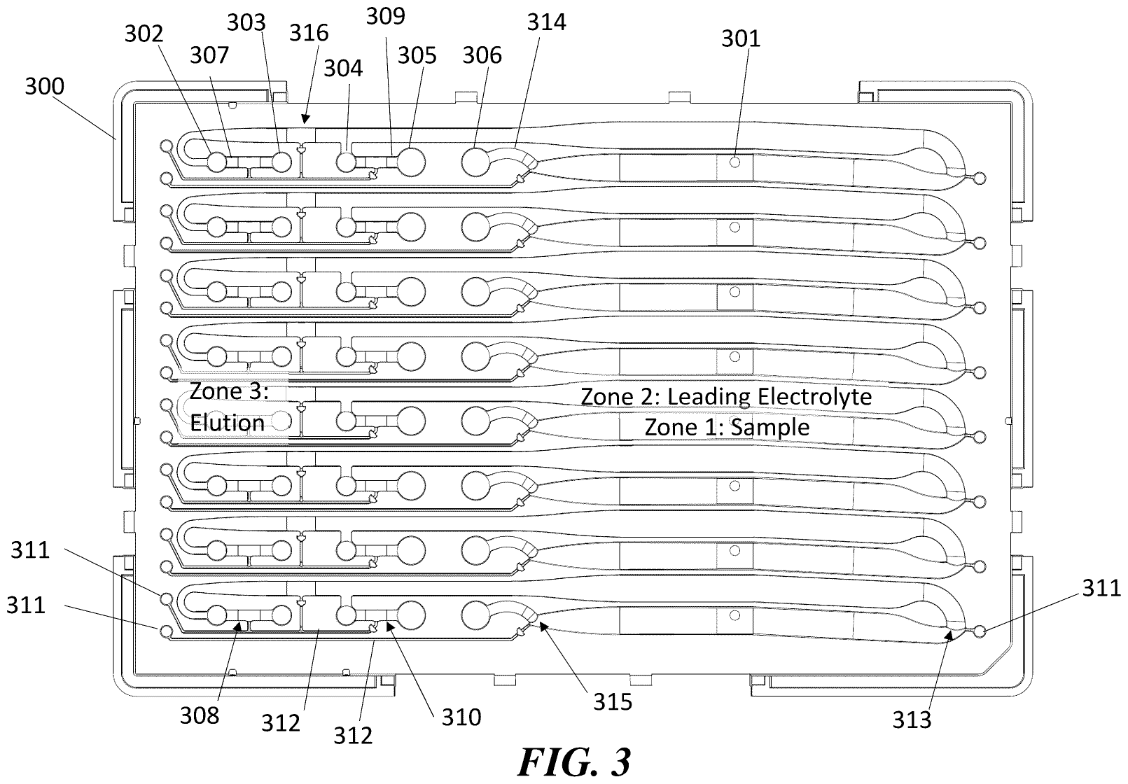

Referring to FIG. 3, the ITP circuit may include a first electrode (e.g., an anode) in a trailing ion buffer well 306, a second electrode (e.g., a cathode) in a leading ion buffer well 305, and a third electrode (e.g., a cathode) in an elution buffer well 303. The elution buffer may have the same components as the leading ion buffer, but with a lower concentration of the leading ion. In a first operation, a voltage may be placed across the first and second electrodes, commencing ITP. As the nucleic acids in the sample approach the branch where the second electrode is positioned, the voltage may be switched between the first and third electrodes. The nucleic acids may then migrate into the branch with the third electrode, to be collected in the elution well 302.

Methods for isotachophoresis are described, for example, in U.S. Pat. No. 8,846,314 (Chambers et al.), 10,233,441 (Santiago et al.), and 10,415,030 (Marshall et al.), each of which is incorporated by reference herein in its entirety. Systems for isotachophoresis are commercially available from Purigen Biosystems, Inc., Pleasanton, CA, USA.

In the case of a sample comprising an ionic detergent, such as SDS, this ionic species may cause deterioration of performance in ITP and downstream assays. However, performance may be improved by contacting the cell lysate with a quencher solution comprising a non-ionic detergent. The non-ionic detergent may comprise a hydrophilic polyethylene oxide chain (e.g., octylphenoxypolyethoxyethanol (“IGEPAL CA-630” or “Nonidet P-40”), a polysorbate (e.g., Tween-20 or Tween 80), Triton X (e.g., Trigon X 100 or Triton X 114), octyl glucoside or octyl thioglucoside), Brij (e.g., Brij S20 or Brij 35), SPAN, pluronic F108, or MEGA. The ratio of non-ionic detergent to ionic detergent in the test sample may be at least about 1:1, at least about 2:1, at least about 3:1, at least about 4:1, at least about 5:1, or at least about 10:1 on a molar basis, e.g., between about 3:1 to about 5:1, e.g., about 4:1.

The quencher solution may further comprise an alkali metal selected from potassium (K), rubidium (Rb), caesium (Cs), and francium (Fr), or an alkaline earth metal selected from beryllium (Be), magnesium (Mg), calcium (Ca), strontium (Sr), barium (Ba), and radium (Ra).

B. Other Methods

DNA may be isolated with silica, cellulose, or other types of surfaces, e.g., Ampure SPRI beads. Kits for such procedures are commercially available from, e.g., Promega (Madison, WI) or Qiagen (Venlo, Netherlands). Nucleic acids may be isolated from a sample by contacting the sample with a solid support comprising moieties that bind nucleic acids, e.g., a silica surface. For example, the solid support may be a column comprising silica or may comprise paramagnetic carboxylate coated beads or a silica membrane. After capturing nucleic acids in a sample, the beads may be immobilized with a magnet and impurities removed. In another method, nucleic acids may be isolated using cellulose, polyethylene glycol, or phenol/chloroform. RNA may be purified using a Qiagen RNeasy kit. Nucleic acids also may be further purified using electrophoresis, e.g., on agarose gels or polyacrylamide gels.

### V. Articles of Manufacture and Systems

The present disclosure also provides articles and systems for performing isotachophoresis.

A system for performing isotachophoresis may include a fluidic device, such as a fluidic chip or cartridge, adapted to engage a cartridge interface of a system.

A cartridge for isotachophoresis may comprise a fluidic channel. The fluidic channel may have a first port positioned to receive a cathode when the cartridge is engaged with the interface of the isotachophoresis system, and a second port positioned to receive an anode when engaged with the interface. Application of a voltage between the cathode and the anode may establish a voltage gradient across the fluidic channel under which ITP may occur.

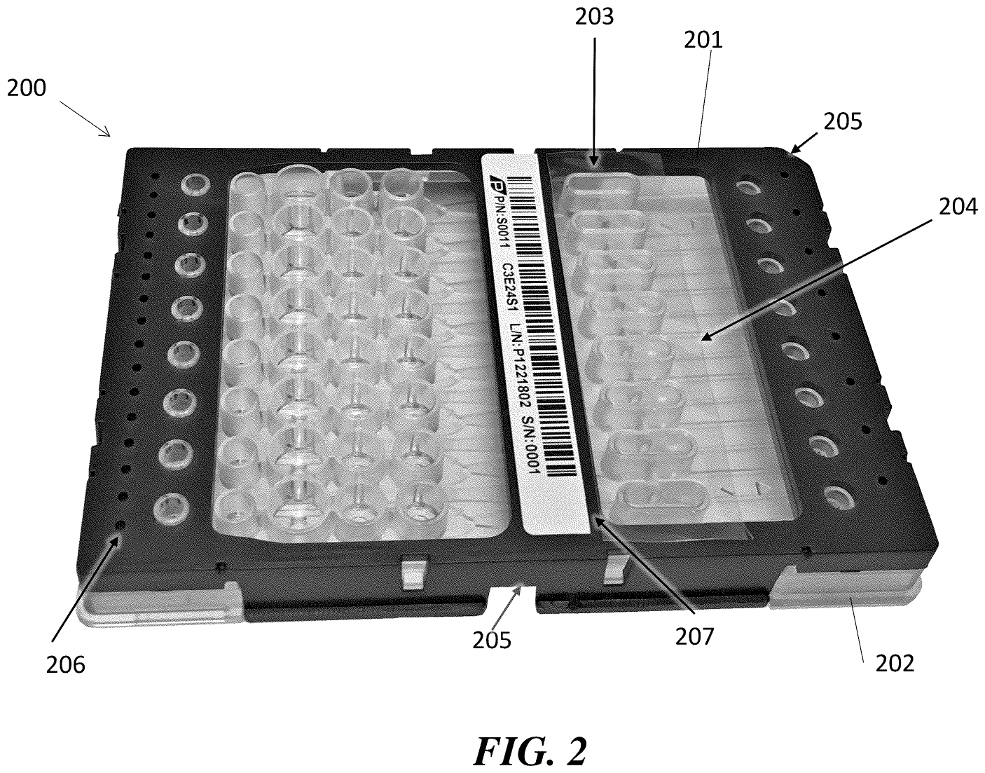

Referring to FIG. 2, an example of a fluidic cartridge 200 includes 8 fluidic channels. The channels include sample inlets 203. FIG. 3 shows the fluidic architecture in more detail. Trailing electrolyte is loaded into well 306. Sample is loaded into well 301. A first leading electrolyte is loaded into wells 304 and 305. A second leading electrolyte, which functions as an elution buffer, is loaded into wells 302 and 303. Pneumatic ports 311 are in pneumatic communication with the fluidic channel, and are positioned between capillary barriers 308, 310, 313 and 315. Electrolyte channels 307, 309 and 314 also guide electrolyte through the fluidic channel. An anode is position in trailing electrolyte well 306, and cathodes are positioned in wells 305 and 303.

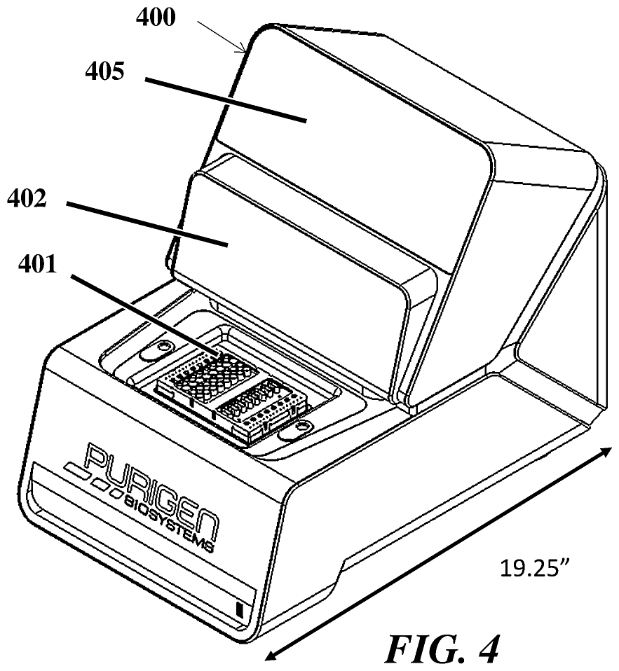

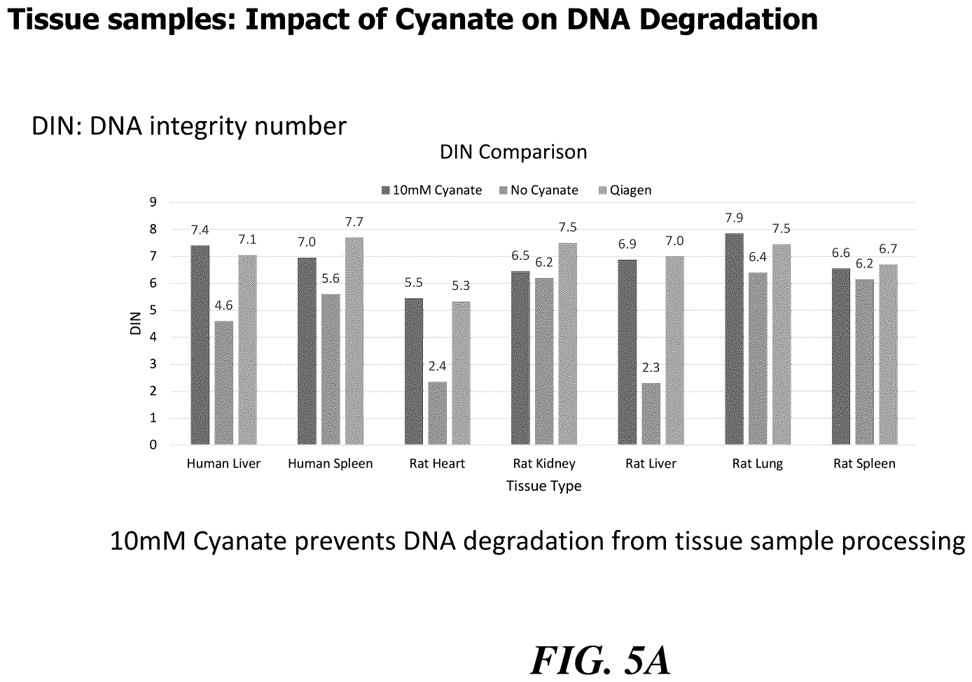

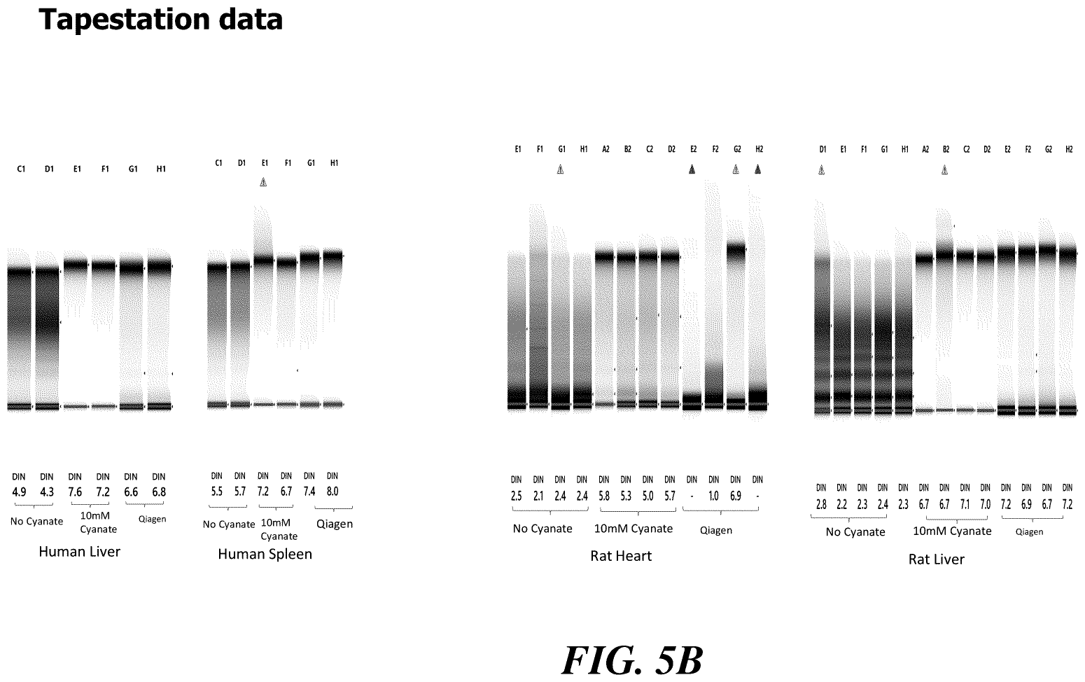

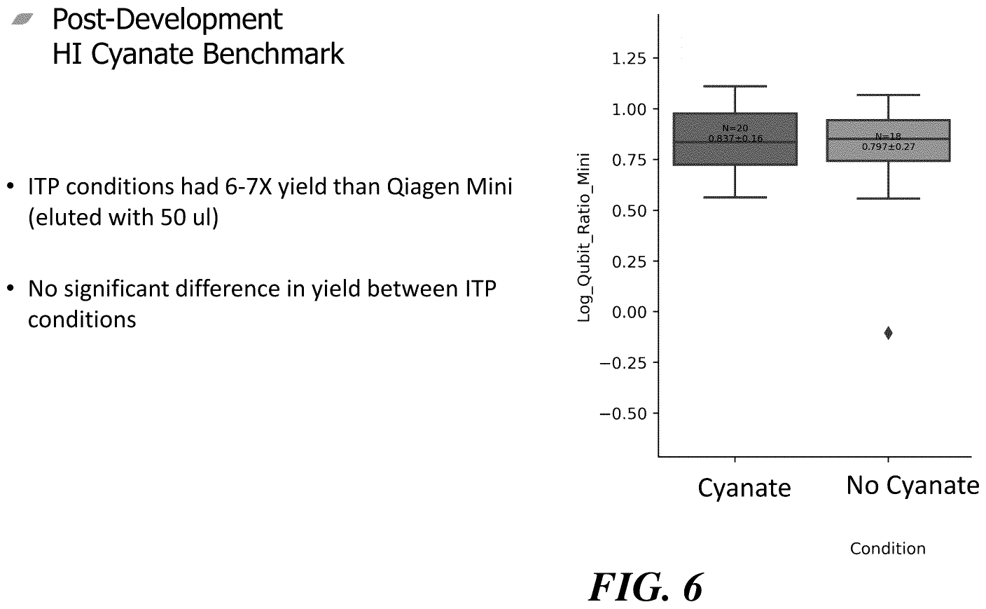

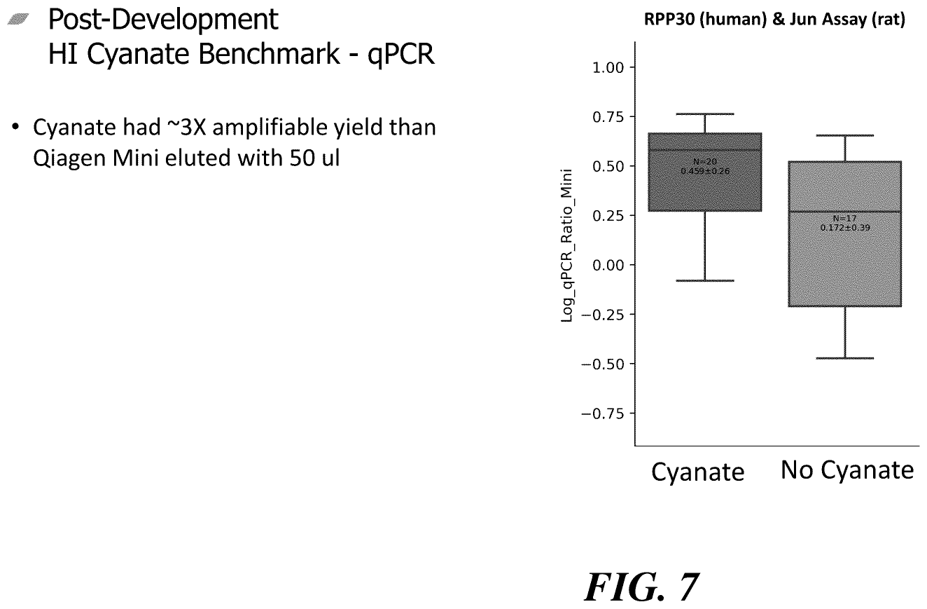

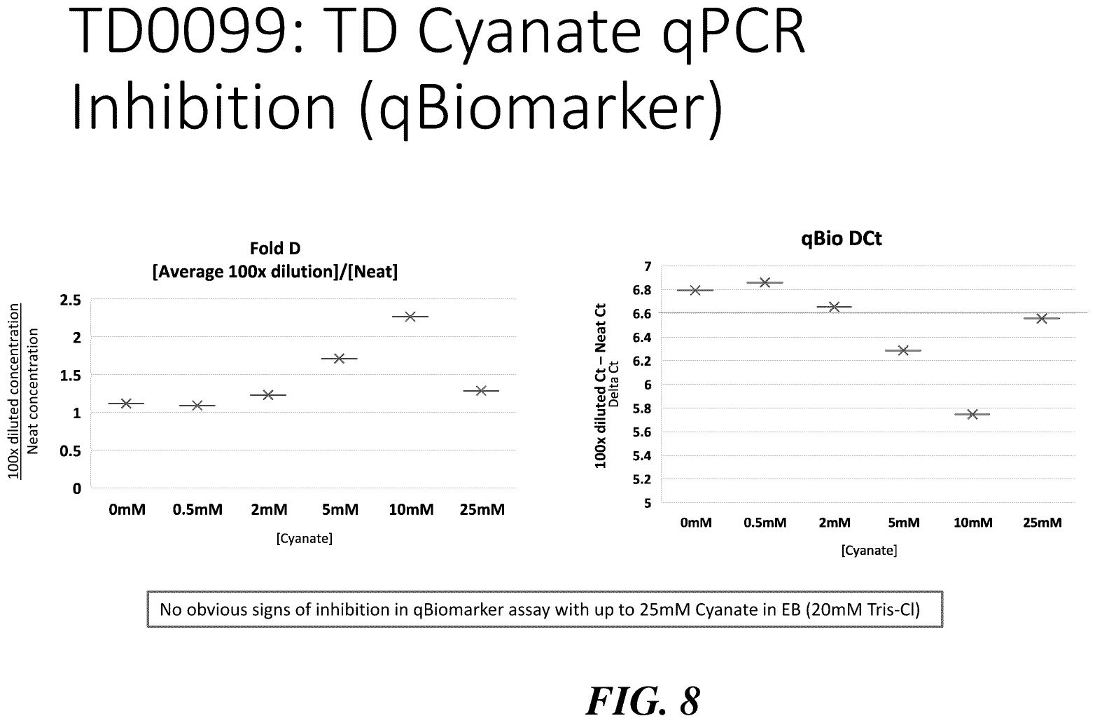

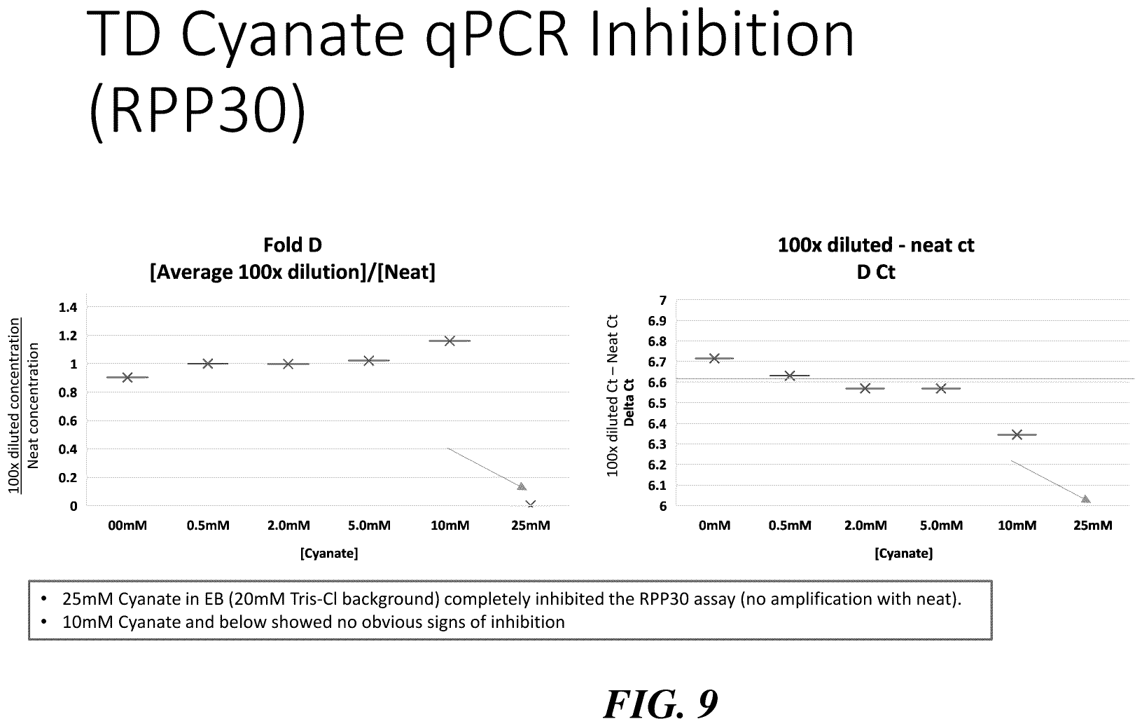

Referring to FIG. 4, instrument 400 includes a cartridge 401 loaded into a cartridge interface. The system 400 may comprise an interface assembly configured to receive and engage the fluidic device pneumatically, electrically and/or fluidically. Touchscreen 405 may function as a user interface. The interface assembly may comprise a key or orientation device to properly orient the fluidic device 401 within the instrument 400. The interface assembly also may comprise electrodes which, when the fluidic device 401 is engaged, may be positioned in various device reservoirs. The interface assembly may further comprise pneumatic lines which, when the device is engaged, may communicate with pneumatic ports in the device 401. The system 400 may include a power supply to apply current and/or voltage to the electrodes. The system 400 may comprise electronics to measure voltage across various electrodes. The system 400 may include pumps to supply positive or negative pneumatic pressure to the pneumatic lines. The system 400 may comprise a temperature sensor to measure temperature at determined positions in fluidic circuits in the device 401. The system 400 may comprise an optical assembly comprising light sources to transmit light to determined positions in the fluidic device and to detect light, e.g., fluorescent light, emitted from the fluidic device, for example, upon excitation of fluorescent species in the device 401. The system 400 may include a display to display operating parameters of the system, such as voltage, temperature, detected light, engagement of a fluidic device, time, stage of processing. The system 400 may be connected through a communications network, such as the internet, to a remote server through which operation of the system may be controlled remotely.

### EXAMPLES

### I. Example 1

An example of a recipe and protocol for DNA purification are:

Lysis Buffer 1:
Tris 15 mM
Sodium Chloride 8 mM
Sodium Cyanate 10 mM
Sodium dodecyl Sulfate (SDS) 0.29%
Dithiothreitol (DTT) 10 mM
Igepal CA-630 0.2%

Lysis Buffer 2:
Tris 15 mM
Sodium Chloride 8.5 mM
Sodium Cyanate 10 mM
Sodium dodecyl Sulfate (SDS) 0.29%
Dithiothreitol (DTT) 10 mM
Igepal CA-630 0.2%

Protocol 1:

185 ul of Lysis Buffer 1 and 10 ul Proteinase K (NEB, P8107S) are added to up to 25 mg fresh frozen tissue and vortexed. The mixture is incubated at 56° C. for 3 hrs with shaking at 1400 RPM. The lysate is spun at 20,000×g for 5 minutes to pellet undigested debris. 170 ul of the lysate is transferred to a new tube. 60 ul Lysis Buffer 2 is added to the lysate as well as 5 ul of RNase. The resulting mixture is incubated at 37° C. for 10 minutes. Nucleic acid is then recovered.

Protocol 2:

185 ul of Lysis Buffer 2 and 10 ul Proteinase K (NEB, P8107S) are added to 1-25 mg fresh frozen tissue and vortexed. The mixture is incubated at 56° C. for 5 hrs while shaking at 1400 RPM. The lysate is spun at 20,000×g for 5 minutes to pellet undigested debris. 170 ul of the lysate is transferred to a new tube. 60 ul Lysis Buffer 2 is added to the lysate as well as 5 ul of RNase. The resulting mixture is incubated at 37° C. for 10 minutes. Nucleic acid is then recovered.

Lysis Buffer 3 (G2 FFPE Lysis Buffer 1):
Tris 15 mM
Sodium Chloride 8.5 mM
Sodium Cyanate 10 mM
Sodium dodecyl Sulfate (SDS) 0.29%
Igepal CA-630 0.2%

### II. Example 2

An example of a recipe and protocol for RNA purification are:

Lysis Buffer:
Tris 50 mM
Sodium Chloride 25 mM
Magnesium Chloride 5 mM
Potassium Cyanate 15 mM
Sodium dodecyl Sulfate (SDS) 0.4%
Dithiothreitol (DTT) 40 mM

Protocol:

Add 130 ul lysis buffer and 20 ul proteinase K (NEB, P8107S) to up to 25 mg fresh frozen tissue and vortex.

Incubate at 37 C for 10 minutes. If mixing is available during heating, mix at 2000 rpm.

Lightly centrifuge the resulting lysate, and proceed to recover nucleic acid.

### III. Example 3

An example of a protocol for quenching SDS before ITP is:

Protocol:

0.1 mg to 25 mg mammalian tissue specimen (fresh-frozen or FFPE) is treated with 200 ul of a lysis buffer containing Igepal-630, SDS, sodium chloride, and sodium cyanate. 1 to ul of ProteinaseK (20 ug-400 ug or 0.8 U-16 U) is added and the mixture is incubated at 37° C.-56° C. for 15 minutes to overnight.

A second buffer is added containing 22% Tween20, a nuclease (DNase for RNA purification and RNase for DNA purification), and, in some cases, divalent cations.

Isotachophoresis is performed on the resulting lysate using a Purigen Ionic System.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

1. A composition comprising:
a) a buffer in aqueous solution;
b) a detergent; and
c) about 5 mM to about 100 mM cyanate or thiocyanate;
wherein a relative concentration of the cyanate or the thiocyanate to urea in the composition is at least about 1:800, at least about 1:600, at least about 1:400, at least about 1:200, at least about 1:10, at least about 1:1, at least about 100:1, at least about 10,000:1, or at least about 1,000,000:1.

2. The composition of claim 1, wherein the detergent is an ionic detergent.

3. The composition of claim 2, wherein the ionic detergent is a cationic, anionic, zwitterionic, or amphoteric detergent.

4. The composition of claim 1, wherein the detergent is a surfactant.

5. The composition of claim 1, wherein the detergent is a non-ionic detergent.

6. (canceled)

7. The composition of claim 1, comprising no more than about 500 mM urea, no more than about 10 mM urea, no more than about 2 mm urea, no more than about 100 nM urea, or no more than about 1 nanomolar urea.

8. The composition of claim 1, wherein the composition is free or essentially free of urea.

9. The composition of claim 1, wherein the composition is free or essentially free of guanidinium.

10. The composition of claim 1, further comprising about mM to 25 mM cyanate or thiocyanate, about 25 mM to 50 mM cyanate or thiocyanate, or about 50 mM to 100 mM cyanate or thiocyanate.

11. The composition of claim 1, wherein the cyanate or the thiocyanate comprises sodium cyanate, sodium thiocyanate, potassium cyanate, or potassium thiocyanate.

12. The composition of claim 1, further comprising an ionic salt.

13-14. (canceled)

15. The composition of claim 12, wherein the ionic salt is present at about 10 mM to 40 mM.

16. The composition of claim 1, wherein the buffer comprises phosphate, Tris, Bis-Tris, e-aminocaproic acid, or HEPES.

17-19. (canceled)

20. The composition of claim 1, wherein the aqueous solution has a pH between about 3.5 and about 9.0.

21. (canceled)

22. The composition of claim 1, wherein the buffer has a concentration between about 10 mM and about 200 mM.

23. (canceled)

24. The composition of claim 1, wherein the detergent comprises a dodecyl sulfate or ethyl trimethylammonium bromide (“ETMAB”).

25. The composition of claim 24, wherein the dodecyl sulfate comprises sodium dodecyl sulfate.

26. The composition of claim 1, further comprising a reducing agent.

27. The composition of claim 26, wherein the reducing agent comprises DTT (dithiothreitol), DTE (dithioerythritol), L-glutathione (GSH), TCEP (Tris (2-Carboxyethyl) phosphine hydrochloride), or beta-mercaptoethanol.

28. The composition of claim 1, further comprising a protease.

29-99. (canceled)
